SEQUENCE LISTING (6 sequences)

SEQ ID NO: 1 - protein, 343 aa, Alcaligenes faecalis
QGAVRKATAAEIAALPRQKVELVDPPFVHAHSQVAEGGPKVVEFTMVIEEKKIVIDDAGTEVHAMAFNGTVPGPLMVVHQDDYLELTLINPETNTLMHNIDFHAATGALGGGGLTEINPGEKTILRFKATKPGVFVYHCAPPGMVPWHVVSGMNGAIMVLPREGLHDGKGKALTYDKIYYVGEQDFYVPRDENGKYKKYEAPGDAYEDTVKVMRTLTPTHVVFNGAVGALTGDKAMTAAVGEKVLIVHSQANRDTRPHLIGGHGDYVWATGKFNTPPDVDQETWFIPGGAAGAAFYTFQQPGIYAYVNHNLIEAFELGAAAHFKVTGEWNDDLMTSVLAPSGT

SEQ ID NO: 2 - protein, 343 aa, Alcaligenes faecalis
MATAAEIAALPRQKVELVDPPFVHAHSQVAEGGPKVVEFTMVIEEKKIVIDDAGTEVHAMAFNGTVPGPLMVVHQDDYLELTLINPETNTLMHNIDFHAATGALGGGGLTEINPGEKTILRFKATKPGVFVYHCAPPGMVPWHVVSGGNGAIMVLPREGLHDGKGKALTYDKIYYVGEQDFYVPRDENGKYKKYEAPGDAYEDTVKVMRTLTPTHVVFNGAVGALTGDKAMTAAVGEKVLIVHSQANRDTRPHLIGGHGDYVWATGKFNTPPDVDQETWFIPGGAAGAAFYTFQQPGIYAYVNHNLIEAFELGAAAHFKVTGEWNDDLMTSVLAPSGTIEGRI

SEQ ID NO: 3 - DNA, 1055 bp, Alcaligenes faecalis
atggcaactgcggcagaaatagcagcacttccacgccagaaggtggagcttgtggaccctcccttcgtgcatgccctagtcaggttgcagaaggcggacccaaggtggtcgaattcaccatggtgatcgaggaaaagaagatcgtcatcgatgatgcgggaaccgaagttcacgccatggcattcaacggcaccgtaccaggaccgctgatggtcgtgcatcaggacgattatctcgaactgacactcatcaaccctgaaaccaacacgctgatgcacaatatcgatttccatgcggcaaccggtgcattgggcggcggcgggctgaccgaaatcaatccgggagaaaagaccatcctgcgcttcaaggcgaccaagcccggcgtcttcgtctaccactgcgcacctcccggaatggttccctggcatgtcgtatcgggcgggaatggtgcgatcatggtgctgccgcgcgagggtctgcatgacggcaaaggcaaagcactgacctacgacaagatttattatgtcggcgaacaggatttctatgtaccgcgcgacgagaacggcaaatacaagaaatacgaggcgcccggcgacgcttatgaagacaccgtcaaggtcatgcgcactctcactccgacccatgtggtgttcaacggcgctgtgggcgcactgactggcgacaaggccatgacggcggcggttggcgagaaagtcctgatcgtccactcgcaggccaaccgcgatacgagaccacatctgatcggggggcatggggattatgtctgggcgaccggcaagttcaatacgccgcccgacgtcgatcaggaaacctggttcattccgggtggtgccgccggagcagccttctacacgttccagcagcccggcatctacgcctatgtgaaccacaatctgatcgaggcttttgaactcggcgctgccgcccacttcaaggtcacgggtgaatggaacgacgatctgatgacgtcggttctcgcaccatctggtacgatcgagggaaggattctcgagcaccaccaccaccaccactga

SEQ ID NO: 4 - DNA, 1055 bp, Alcaligenes faecalis
atggcaactgcggcagaaatagcagcacttccacgccagaaggtggagcttgtggaccctcccttcgtgcatgccctagtcaggttgcagaaggcggacccaaggtggtcgaattcaccatggtgatcgaggaaaagaagatcgtcatcgatgatgcgggaaccgaagttcacgccatggcattcaacggcaccgtaccaggaccgctgatggtcgtgcatcaggacgattatctcgaactgacactcatcaaccctgaaaccaacacgctgatgcacaatatcgatttccatgcggcaaccggtgcattgggcggcggcgggctgaccgaaatcaatccgggagaaaagaccatcctgcgcttcaaggcgaccaagcccggcgtcttcgtctaccactgcgcacctcccggaatggttccctggcatgtcgtatcgggcatgaatggtgcgatcatggtgctgccgcgcgagggtctgcatgacggcaaaggcaaagcactgacctacgacaagatttattatgtcggcgaacaggatttctatgtaccgcgcgacgagaacggcaaatacaagaaatacgaggcgcccggcgacgcttatgaagacaccgtcaaggtcatgcgcactctcactccgacccatgtggtgttcaacggcgctgtgggcgcactgactggcgacaaggccatgacggcggcggttggcgagaaagtcctgatcgtccactcgcaggccaaccgcgatacgagaccacatctgatcggggggcatggggattatgtctgggcgaccggcaagttcaatacgccgcccgacgtcgatcaggaaacctggttcattccgggtggtgccgccggagcagccttctacacgttccagcagcccggcatctacgcctatgtgaaccacaatctgatcgaggcttttgaactcggcgctgccgcccacttcaaggtcacgggtgaatggaacgacgatctgatgacgtcggttctcgcaccatctggtacgatcgagggaaggattctcgagcaccaccaccaccaccactga

SEQ ID NO: 5 - DNA, 32 bp, Artificial Sequence
  Standard forward primer
catggtgctgccgcgggagggtctgcatgacg

SEQ ID NO: 6 - DNA, 60 bp, Artificial Sequence
  M150G reverse primer
gccgtcatgcagaccctcccgcggcagcaccatgatcgcaccattcccgcccgatacgac

(19) United States
(12) Patent Application Publication
(10) Pub. No.: US 2010/0167311 A1
(43) Pub. Date: Jul. 1, 2010

(54) Oxidoreductases and Processes Utilising Such Enzymes

(76) Inventors: Gerard W. Canters, Leiden (NL); Hein Jakob Wijma, Durham, NC (US); Michael Murphy, Vancouver (CA); Iain Macpherson, Vancouver (CA)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/223,850

(22) PCT Filed: Feb. 8, 2007
(86) PCT No.: PCT/EP2007/051232

§ 371 Date: Jul. 27, 2009

(30) Foreign Application Priority Data
Feb. 9, 2006 (EP) 06250703.3

(51) Int. Cl. G01N 33/573; C12N 9/02; C07H 21/00; C12N 1/00; C12M 1/34

(52) U.S. Cl. 435/7.4; 435/189; 536/23.2; 435/243; 536/23.1; 435/287.1

(57) ABSTRACT

In Cu-containing nitrite reductase from Alcaligenes faecalis S-6 the axial methionine ligand of the type 1 site was replaced (M150G) to make the copper atom accessible to external ligands that might affect the enzyme's catalytic activity. The type-1 site optical spectrum of M150G (A460/A600=0.71) differs significantly from that of the native nitrite reductase (A460/A600=1.3). The reduction potential of the type-1 site of nitrite reductase M150G (EM=312−5 mV versus hydrogen) is higher than that of the native enzyme (EM=213−5 mV). M150G has a lower catalytic activity (kcat=133−6 s−1) than the wild-type nitrite reductase (kcat=416−10−s 1). The binding of external ligands to M150G restores spectral properties, reduction potential (EM<225 mV), and catalytic activity (kcat=374−28 s−1). Also the M150H (A460/A600=7.7, EM=104−5 mV, kcat=0.099−0.006 s−1) and M150T (A460/A600=0.085, EM=340−5 mV, kcat=126−2 s−1) variants were characterized to compare their properties with those of M150G. Crystal structures show that the ligands act as allosteric effectors by displacing Met62 which moves to bind to the Cu in the position emptied by the M150G mutation. The reconstituted type-1 site has an otherwise unaltered geometry. The observation that a rearranged ligand can introduce allosteric control in a redox enzyme suggests potential for structural and functional flexibility of copper-containing redox sites.

The present invention relates to electron transfer enzymes derived from wild-type oxidoreductases having a type 1 copper site, engineered to replace an axial or equatorial co-ordinating amino acid residue by another residue. The activity of the enzyme may be affected using allosteric solute molecules. This allows the presence or level of solute analytes to be determined using electrodes.

Nature often uses copper to mediate electron transfer in biological redox chains. For this purpose the copper is incorporated in a protein scaffold in a mononuclear so-called type-1 site or in a closely related dinuclear Cu_{A} site (1). These sites can be found throughout the kingdoms of life, from archaea to humans. In photosynthesis and respiration small type-1 site containing proteins (cupredoxins) shuttle electrons between larger enzymes. In enzymes, type-1 (or Cu_{A}) sites enable electron transfer between catalytic sites and external electron donors. These enzymes are often involved in respiration (nitrite reductase, cytochrome c oxidase) or in the conversion of metabolites (multi-copper oxidases).

The physiological role of NiR is the dissimilatory reduction of nitrite (NO₂⁻+2H⁺+e⁻→NO+H₂O) (2) although NiR does catalyze bidirectionally; at pH 8 the k_{cat} of the reverse reaction is higher than the k cat of the forward reaction (3). NiR is a homotrimer, in which each subunit contains a type-1 copper site that transfers electrons from a physiological electron donor to a type-2 copper site that is located deeper inside the enzyme (46). The type-2 copper forms the active site together with a water network and an Asp-His pair, that bind the nitrite and donate protons (7-10).

In a type-1 site, two histidines and one cysteine bind the copper; these three ligands are very strongly conserved. In addition, one or two weaker binding, axial ligands can be present. A methionine or a glutamine can serve as the fourth (axial) ligand and sometimes a fifth axial ligand, in the form of a backbone carbonyl oxygen from glycine, can bind on the opposite side (11). The two-histidines/one-cysteine ligand set (His 95, Cys 136, His 145) results in unique spectroscopic properties of the oxidized type-1 site (Cu²⁺; the Cu¹⁺ state is spectroscopically silent). All characterized type-1 copper sites have a unique small hyperfine splitting in their EPR spectra (11). Furthermore, strong absorption bands at approximately 600 nm and often also around 460 nm result in a blue or green color, depending mostly on the binding geometry of the weaker axial ligands. In this specification, we will refer to these absorption bands as 460 and 600 nm bands also when they are slightly shifted.

One approach to study the function of a ligand in a metal site is to engineer the ligand out and add external ligands that may bind in the gap created in the first coordination shell (12). The interesting question is then how the binding of the external ligand affects the properties of the metal site. Earlier this approach was used to investigate the type-1 copper site in azurin (12-19). By using the enzyme nitrite reductase, it is possible to monitor if the type-1 site is functioning since a functional type-1 site is necessary for the catalytic activity. For NiR, we earlier found (20) that when this approach was applied to the C-terminal histidine ligand, catalytic activity was lost because the midpoint potential of the type-1 site was altered too much, also in the presence of external ligands. Because axial ligands less drastically influence the reduction potential of the type-1 site than the equatorial ligands (11,21-28), we investigated whether in such an axial cavity variant the electron transfer function could be better restored by external ligands. This question was not addressed in earlier reports (18,29,30). The structure of type-1 sites in general consists of an N-terminally located histidine that is part of an internal loop connecting two beta-strands, and three C-terminally located residues, a cysteine, a histidine and finally a methionine. These latter three residues are located on another loop.

The ligands by which the Cu is bound in the type-1 site are His95, Cys136, His 145 and Met150 (numbering is according to the NiR from Alcaligenes cycloclastes S-6). The Met 150 coordinates as an axial ligand while the two His and one Cys residue coordinate equatorially. In other blue copper proteins the axial ligand may be glutamine, valine or leucine. There may be additional weaker coordinate from a second axial ligand, for instance from the carbonyl group of a residue such as glycine.

According to the invention there is provided a new method of detecting redox enzyme activity in which an electron transfer enzyme derived from a wild type oxidoreductase having a type-1 copper site is contacted with a substrate for the electron transfer to oxidise or reduce the substrate and the enzyme activity is monitored via the activity of an oxidant or reductant as the case may be of the type-1 copper site, characterised in that the type 1 copper site has been modified compared to the wild type enzyme by substitution of a copper coordinating residue which coordinate the copper ion of the type 1 site by a residue selected from Gly and Ala, and the enzymatic reaction is carried out in the presence of an allosteric effector which is a solute molecule which is capable of modifying the activity of the enzyme to allow an electron donating residue of the enzyme to coordinate with the copper ion of the type 1 copper site.

The invention is of most benefit where there is electron transfer between the electron transfer protein and an electrode—that is, where the oxidant or reductant for the type 1 site is an electrode directly or via a separate electron transfer site of the protein, for instance a Cu type 2 site, and/or via redox mediators in solution and/or via redox partners (separate proteins with redox centres which may be immobilised with the enzyme). By measuring the current, or alternatively the electrical resistance, between electrodes in contact with the protein, the level of enzyme activity can be determined. Alternatively the progress of the reaction may be monitored spectrophotometrically using a chromogenic or fluorogenic substrate for the enzyme, i.e. which has a different spectrum in oxidised and reduced forms. Thus the oxidant or reductant is a second substrate for the redox enzyme, which is reduced or oxidised at a different active site to the first substrate.

The invention is based on the observation that replacement of an axial methionine ligand of a type 1 site by a small residue, preferably glycine, activity of the enzyme is reduced by 60 to 70%. The type 1 site is crippled by the mutation and does not function optimally anymore. The mutation also creates a gap in the protein structure since the glycine that replaces methionine only has hydrogen atom as the side chain, while the side chain of a methionine residue is voluminous.

We have observed that there is a neighbouring methionine (Met62, according to the numbering system of NiR from A. cycloclastes) in the structure, that in the mutated enzyme can move and bind at the position of the deleted methionine and thereby restore full activity of the enzyme. In other words the gap created by the mutation is filled by Met62 but the movement of Met62 in its turn creates a new hole in the structure. The crucial finding is that this movement of Met62 only occurs when there are small solute molecules in the reaction mixture which are able to fill the cavity created by the Met62 movement. Thus, when a small molecule is present, the mutated protein recovers its activity while in the absence of such a molecule the enzyme will have lost most of its activity. The small molecule acts as an allosteric effector.

In the invention the allosteric effector does not interact directly with the type 1 copper site, nor with the enzyme's active site, but rather with a site remote from these regions which affects the enzyme activity.

Thus the residue which is mutated is preferably an axial residue, e.g. glutamine, valine, leucine, or preferably methionine residue. It is possible that the same effect may be achieved where one of the equatorial ligand residues is mutated and in another embodiment the residue which is mutated is an equatorial Cys or His ligand. The electron donating residue which becomes coordinated with the copper ion is preferably methionine but may be cysteine, histidine, glutamine or serine.

In the invention electron transfer to and from an electrode may be by direct contact of the enzyme with the electrode or via electron transfer proteins or mediators. Where the contact is direct, the enzyme may be immobilised onto the electrode, for instance by known immobilisation techniques, ensuring that the enzyme remains active and electron transfer to and from the catalytic site via the copper type 1 site to the electrode is possible. Electron transfer from the copper type 1 site of an immobilised enzyme may be direct to the electrode or via another redox site in the protein, preferably via another copper site, for instance a type 2 copper site.

The invention may be used with any blue copper oxidoreductase enzymes. Examples of enzymes having a type 1 copper site include large blue copper proteins such as the blue oxidases, e.g. laccase, ascorbate oxidase, ceruloplasmin and Fet3p. Preferably the electron transfer enzyme is based on an oxidoreductase which is a dissimilatory nitrite reductase, most preferably based on NiR from A. faecalis S-6.

The essential mutation from the wild type oxidoreductase is that an axial or equatorial ligand residue is replaced by a small residue such as glycine or alanine. Where the wild type enzyme contains multiple copper sites, the sites are preferably also included as part of the electron transfer enzymes activity. However it may be possible to mutate out these other copper electron transfer sites, provided that electron transfer to an oxidant or reductant, e.g. the electrode, may still take place and the protein is still catalytically active.

The enzyme is preferably derived from the wild-type enzyme having sequence ID1, which is nitrite reductase from A. faecalis. The enzyme may have up to 10 residues from the C and or N terminals deleted. The residue which is substituted by Ala or Gly is selected from His95, Cys136 and Met 150, and is preferably Met150. The other three of these residues are unchanged. The remaining residues include at least one electron donating residue, preferably Met, residue which is unchanged from wt and which can, in the folded conformation, coordinate with the type 1 copper site. Preferably the Met62 residue which is unchanged. The remaining residues may be conservatively substituted or deleted, but preferably at least 50% are identical to those of sequence ID1, more preferably at least 75%, most preferably at least 90% of the remaining residues are identical to that of sequence ID1.

A particularly preferred enzyme has sequence ID2.

In the wild-type enzyme electrons will be transferred from one substrate to another compound so that the cycle of oxidation and reduction can continue. Nitrite reductase reduces nitrite (the substrate) to NO using an electron which is provided from some source and is the reductant.

In the cell the reductant is an electron transfer protein, i.e. pseudo-azurin, in vitro it can be any electron-rich compound (reductant) like ferrocyanide, methylviologen or an electrode. To be able to use the mutated NiR as a sensor, in the invention both sides of the chain should be operational, i.e. there should be nitrite in the sample and there should be a reductant (like pseudo-azurin or viologen that can be monitored optically, or an electrode that can be monitored electrochemically) that is able to reduce the type-1 site. When the enzyme is activated by the allosteric compound nitrite is converted while the reductant is oxidized. The progress of the reaction is observed optically or as an increase in current.

The electron transfer enzyme has a catalytic site for oxidation or reduction of at least one substrate. In the invention substrate is supplied to allow turnover to take place during enzyme activity. Examples of substrates include pseudoazurin, substrate for NiR from A. faecalis. Nitrite is also supplied to allow enzyme turnover.

In the invention, the solute molecule acts as an allosteric effector for the electron transfer enzyme. The protein may be engineered so as to have a specific binding site, to enable detection of the molecule for which the site is specific. In the invention it is preferred that a range of electron transfer proteins are engineered, each with different specific binding sites for different solute molecules. Such an array of proteins may be utilised in a sensor having an array of electrodes to provide an enzyme activity profile, thereby allowing identification of solute present in a given sample.

Solute molecules which would usefully act as the allosteric effector to be detected using the invention include metabolites, such as creatinine, cholesterol, drugs, hormones, sugars, fatty acids, peptides, as well as other analytes such as alcohols, imidazoles, acetamide, dimethylsulfide and other sulfides such as ethyl methyl sulfide.

According to a further aspect of the invention there is provided a new sensor comprising an electrode and, in contact with the electrode, a reaction medium containing:—
1) an electron transfer enzyme derived from a wild-type oxidoreductase having a type 1 copper site, that has been modified as compared to the wild-type enzyme by substitution of a copper coordinating ligand residue which coordinates the copper ion of the type 1 copper site by a residue selected from Gly and Ala;
ii) a substrate for the electron transfer enzyme; and
iii) an allosteric effector, or a sample suspected of containing the allosteric effector, which is a solute molecule, that is capable of modifying the activity of the enzyme to allow an electron donating residue of the electron transfer enzyme to coordinate the copper ion of the type 1 copper site.

Preferably the sensor is provided in a form such that addition of a sample creates a reaction medium containing the necessary components. Thus the kit may comprise electrodes each in a vessel containing the enzyme and the substrate. Preferably the sensor is suitable for connection into a circuit which contains current or resistance measuring and recording means.

Where a sensor comprises an array of electrodes, each carrying separate proteins, it is most convenient for a single aliquot of sample suspected of containing the aliquot to be applied substantially simultaneously or at least in parallel with all of the electrodes.

The present invention is further illustrated in the accompanying examples.

Materials and Methods

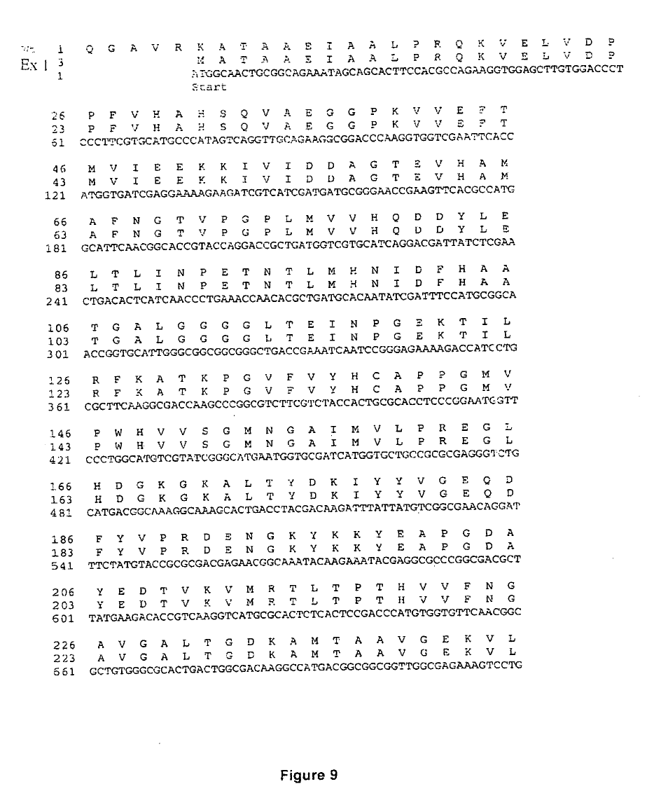

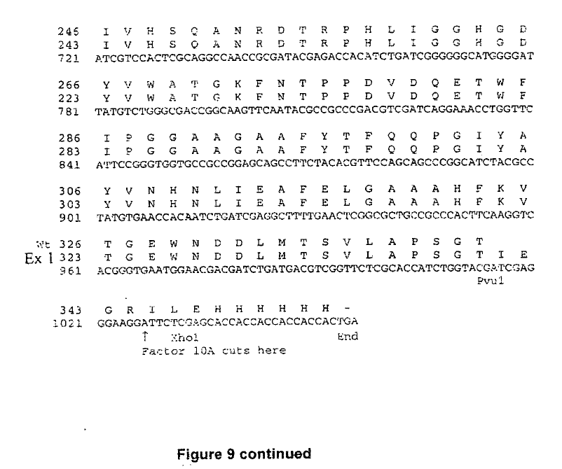

Materials—For mutagenesis and expression a pET28b based vector (7) containing nirK from A. faecalis S-6 (42) was used. The sense sequence is shown in sequence ID4. This omits a leader sequence and additional 6 residues at the N terminal of wt NiR and which includes extra residues at the C terminal including a His tag and factor Xa recognition site. FIG. 9 compares this sequence used with wt NiR published elsewhere. The NiR gene is inserted into pET28b involving the Xho1 site. The second site is unclear. There are 3 Nco I sites close to each other on the N-terminal side. It can be seen from FIG. 9 that the first 5 amino acids from the N-terminal end of the wt protein are omitted. The first amino acid in the cloned gene is called number 3. At the C-terminal end of the protein there are 7 additional amino acids. Five of which remain after removal of the His-tag with Factor X^{a}. By adding those amino acids at the C-terminal end a Pvu1 site was created. For introduction of the mutations the following primers were used together with standard molecular biology techniques; standard forward primer, CAT GGT GCT GCC GCG GGA GGG TCT GCA TGA CG (sequence ID5); M150G reverse primer, GCC GTC ATG CAG ACC CTC CCG CGG CAG CAC CAT GAT CGC ACC ATT CCC GCC CGA TAC GAC (sequence ID6) (underlined is a Sac II restriction site that was introduced by a silent mutation; in bold are the altered bases of which CCC is the antisense codon for Gly; for M150H the alteration was GTG, for M150T it was CGT). Expression and purification of NiR, and of its physiological electron donor pseudoazurin (43-45), were achieved as described previously (3,20).

For crystallography and for activity assays a gel-filtration step was added as the last step of the purification of NiR as described (3). The Cu-content determined with bicinchoninic acid (46) was 1.9 for wt NiR, 1.7 for NiR M150T, 1.7 for NiR M150H (quoted numbers are per monomer), and 1.0 for pseudoazurin. For NiR M1500 the Cu-content varied between 1.7-2.1 per batch; a batch with a Cu-content of 1.9 was used for the activity assays and for crystallization.

Spectroscopy and assays—The spectrophotometer was a Perkin Elmer Instruments Lambda 800. Prior to measuring spectra, samples were spun down at 16,000 g for 10 minutes to remove small quantities (<5%) of aggregated protein that in the case of NiR can produce a scattering contribution comparable in intensity to the absorption spectrum of the type-1 site. NiR M150G (50 μM) was titrated with ligands in 50 mM Mops pH 7.0. After correction for dilution both the increase of absorbance (A) at 460 nm, and the decrease at 600 nm were least-squares fitted assuming a single binding site (A=A^{NoLigand}+ΔA·[L]/(K_{D}^{OX}+[L], in which L is the free ligand concentration). For all the assays in the presence of ligands, the total ligand concentration exceeded the protein concentration at least 10-fold and is therefore taken as equal to the free ligand concentration.

Activity assays were carried out by monitoring the oxidation of pseudoazurin as described (3). The concentrations of the electron donor pseudoazurin (275-325 μM) and the electron acceptor nitrite (5 mM) were saturating. The concentration of NiR was typically 1 nM. The buffer for activity assays was always 50 mM Mops pH 7.0. Whenever using volatile compounds, the cuvette was sealed with a PTFE stopper. All reported activities were calculated from initial rates. Apparent dissociation constants (K_{D}^{app}) were obtained from a least-squares fit of activity (v) versus ligand concentration to v=v^{NoLigand}+Δv×[L]/(K_{D}^{app}+[L]). The meaning of K_{D}^{app} will be explained in the Discussion section.

Potentiometric titrations—Potentiometric titrations were carried out as described by Dutton (47) in a cuvette held at 298 K in 100 mM potassium phosphate pH 7.0. The NiR concentration was typically 40 mM. Diaminodurol (2,3,5,6-tetramethyl-1,4-phenylenediamine) was used as a redox mediator at 100-200 μM. Potassium ferricyanide and sodium dithionite were used to change the potential of the solution. Visible absorption and the potential of the solution were monitored until both were stable. Spectra were recorded in the range of 510-800 nm since diaminodurol gives negligible absorbance in this region. For the M150H mutant, phenazine methosulfate (N-methyldibenzopyrazine methyl sulfate, 10 μM) was used as a redox mediator while the scan range was 400-800 nm. The absorption of oxidized NiR M150H (30 μM) exceeded that of the phenazine methosulfate tenfold.

The recorded spectra were integrated using a routine written in Igor Pro (WaveMetrics Inc.). For base line correction this routine approximated the scattering contributions (due to aggregated protein) either by a linear approximation or by a method described elsewhere (48). There was no need to correct for the type-2 site contribution since the absorption of the type-2 site in this part of the spectrum is 30 times lower than that of the type-1 site (20). The integrated absorbance versus potential was fitted to the Nernst equation with the number of electrons held at one. Therefore, the midpoint potential versus ligand concentration was fitted to equation 1 (49),

E_{M}=E_{M}^{NL}−(RT/F)ln [K_{D}^{red}×(K_{D}^{ox}+[L])/(K_{D}^{ox}(K_{D}^{red}+[L]))] (1)

in which E_{M}^{NL} is the reduction potential without ligand, [L] denotes the free ligand concentration, K_{D}^{ox} and K_{D}^{red} are the ligand dissociation constants from the oxidized and reduced type-1 site respectively, R is the gas constant, F is the Faraday constant and T is the absolute temperature. Because the ligand concentration far exceeded the protein concentration, [L] was set equal to the total ligand concentration. The midpoint potential of the type-1 site with the external ligand bound (E_{M}^{L}) was calculated from equation 2.

E_{M}^{L}=E_{M}^{NL}−(RT/F)ln [K_{D}^{red}/K_{D}^{ox}] (2)

A series of control experiments (47) were carried out to exclude artifacts due to the binding of a redox mediator, oxidant or reductant to the protein. The midpoint potentials of M150G and wt NiR were also determined in the absence of diaminodurol using higher concentrations of ferro/ferricyanide (1-10 mM) as redox mediator, which gave identical results. Replacing sodium dithionite with L-ascorbic acid gave an identical midpoint potential for the wt NiR, but slower equilibration. When TMPD and DCPIP were used as redox-mediators, as has been done for NiR from Rhodobacter sphaeroides (26), identical results were obtained. However, we preferred not to use the latter two mediators since they absorb in the same spectral region as nitrite reductase, and resulted in a slower equilibration. For every midpoint potential here reported, both a reductive and an oxidative titration was carried out. They resulted in the same midpoint potentials. We could not detect significant differences in the midpoint potential of fully Cu-loaded M150G (2.0 Cu per monomer) and partially type-2 depleted batches (1.7 Cu per monomer). The potential of the reference electrode was calibrated with quinhydrone [0.2 g in 10 ml of 100 mM phosphate buffer pH 7.0 gives a solution potential of 286 mV versus the normal hydrogen electrode (NHE)].

Structure determination—Met150Gly crystals were grown at room temperature by the hanging drop vapor diffusion method. The crystallization conditions were 10 mM sodium acetate pH 4.5, 2 mM zinc acetate, 2 mM cupric sulfate, 60-100 mM ammonium sulfate, and 4-10% poly(ethylene glycol) 6000. A stock protein concentration of 35 mg/ml in 10 mM Tris pH 7 was used. These conditions resulted in blue crystals that grew in an orthorhombic lattice (space group P212121). Once grown, crystals were soaked in mother liquor containing either 2 mM dimethylsulfide (DMS) or 200 mM acetamide until they turned from blue to green indicating an alteration of the type-1 copper site. Crystals were then transferred to mother liquor supplemented with glycerol as a cryoprotectant and either DMS or acetamide. DMS-soaked crystals were looped into a cryostream (Oxford Cryo Systems) for home source diffraction studies using a MAR345 detector and Rigaku RU-300 x-ray generator. Acetamide-soaked crystals were looped and immersed in liquid nitrogen for data collection using a MAR345 detector at the Stanford Synchrotron Radiation Laboratory (beamline 7-2). Both DMS and acetamide-soaked crystals diffracted to greater than 1.8 Å resolution and diffraction data was processed with DENZO (50).

DMS and acetamide-soaked M150G crystals contain the NiR trimer in the asymmetric unit. A 1.4 Å resolution structure of nitrite-soaked wt NiR (51) was used as the starting refinement model after removal of the Met150 side-chain, nitrite and selected waters. The structures were refined using REFMAC (52) with 5-7% of the data set aside for calculation of the free R-factor. Fo-Fc difference maps were used to locate the acetamide and DMS ligands and to define the conformation of Met62. The copper ligand geometry and positions of the copper atoms were not restrained throughout the refinement. Each chain of both structures begins at Ala4 and ends at Gly339. At least 90% of the residues in each structure occupy the most favorable position in the Ramachandran plot as described by PROCHECK (53). Statistics of data processing and structure refinement are presented in Table 1.

TABLE 1
Crystallographic Data Collection and Refinement Statistics

| | Crystal | | | |
|---|---|---|---|---|
| | M150G | | M150G | |
| | Dimethylsulfide | | Acetamide | |
| cell dimensions (Å) | a = 61.97 | | a = 61.40 | |
| | b = 103.0 | | b = 102.4 | |
| | c = 146.0 | | c = 146.3 | |
| resolution (Å) | 1.80 | (1.85-1.80)^{A} | 1.60 | (1.64-1.60) |
| r-merge | 0.068 | (0.292) | 0.098 | (0.320) |
| {/}/{σ(/)}^{B} | 22.1 | (6.43) | 10.8 | (3.15) |
| Completeness (%) | 86.5 | (93.8) | 83.0 | (82.0) |
| unique reflections | 76078 | (8146) | 100959 | (9877) |

| | | |
|---|---|---|
| working R-factor | 0.166 | 0.177 |
| free R-factor | 0.199 | 0.209 |
| rmsd bond length (Å) | 0.009 | 0.008 |
| overall B-factor (Å²)^{C} | 19.8 | 25.9 |
| water molecules | 1165 | 1158 |
| PDB entry code | | |

^{A}Values in parenthesis are for the highest resolution shell.
^{B}{/}/{σ(/)} is the average intensity divided by the average estimated error in intensity.
^{C}B-factors are an average from all three monomers.

Results

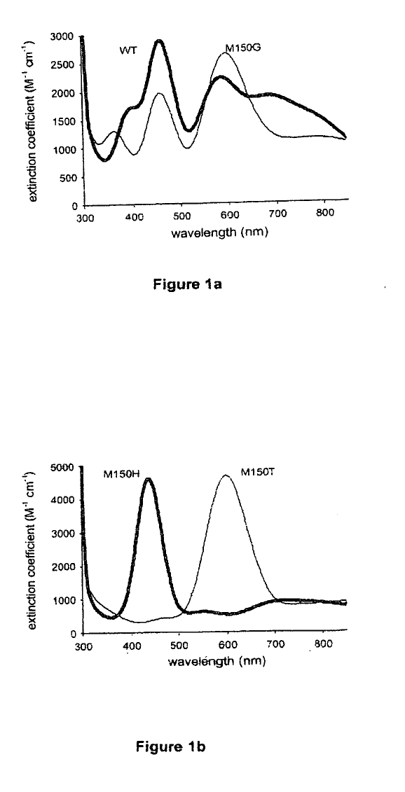

Spectral Characterization and Binding of External Ligands—Purified NiR M150G appeared to the eye as blue, unlike wt NiR which is green. FIG. 1 shows the optical spectra of native and mutant nitrite reductases. (A) NiR wt and NiR M150G. (B) NiR M150H and M150T. Notice the different vertical scale in both panels. A UV/Vis spectrum (FIG. 1A) shows that the blue color is the result of a change in the relative contributions of the absorption bands around 460 and 600 nm (NiR wt ε₄₆₀=2900 M⁻¹ cm⁻¹, ε₅₈₉=2200 M⁻¹ cm⁻¹; NiR M150G ε₄₅₇=2000, ε₆₀₀=2800 M⁻¹ cm⁻¹). Two additional mutants were produced as controls, one for “strong axial interaction” (imidazole side-chain in M150H) and one for “weak axial interaction” (alcohol side-chain in M150T). For NiR M150H and NiR M150T the visible spectrum did differ significantly from the wt NiR spectrum (FIG. 1B). In NiR M150H the 460 nm band has gained in absorption and is shifted to significantly shorter wavelengths, while a weak absorption is visible at 547 nm (ε₄₃₉=4600 M⁻¹ cm, ε₅₄₇=600 M⁻¹ cm⁻¹). For M150T almost all absorption is present in the 600 nm band (ε₄₆₀=400 M⁻¹ cm⁻¹, ε₆₀₂=4700 M⁻¹ cm⁻¹). As a result of the spectral changes NiR M150H is yellow and NiR M150T is blue.

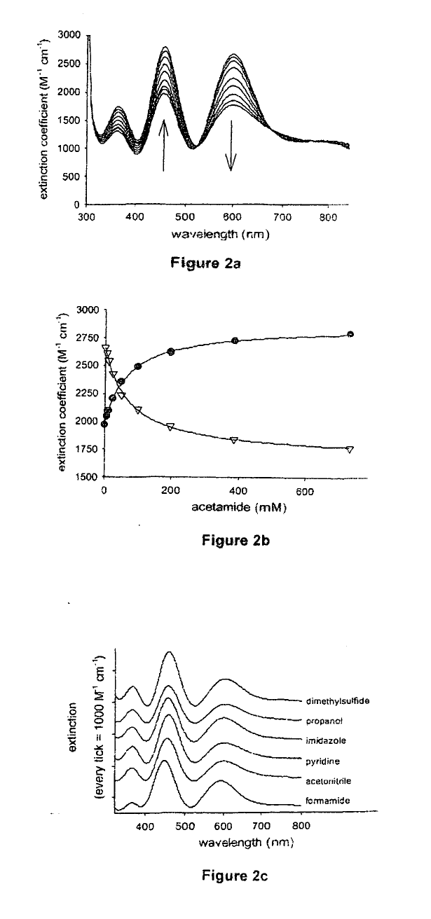

To study ligand binding to the oxidized type-1 site of M150G we monitored the optical spectrum upon addition of different compounds. FIG. 2 shows the optical spectra on titration of NiR M150G with external ligands. (A) Effect of acetamide on the optical spectrum of NiR M150G. Arrows indicate the direction of the spectral changes occurring upon subsequent additions of acetamide. (B) The absorption at 600 (open triangles) and 460 nm (filled circles) plotted versus acetamide concentration. The lines are from fits to a single binding site as described in the Materials and Methods section. (C) Optical spectra, shifted vertically with respect to each other, of NiR M150G with several external ligands. The concentrations were: dimethylsulfide, 151 mM (=7×K_{D}); propanol, 1940 mM (=5.5×K_{D}); imidazole, 250 mM (=5×K_{D}); pyridine, 30 mM (=11.5×K_{D}); acetonitrile 0.1036 mM (=14.5×K_{D}); formamide 850 mM (=5×K_{D}). Ligands of a great variety all caused a stronger absorption at 460 nm, and a weaker absorption at 600 nm. Isosbestic points were observed, and the titration data could be fit to a single binding site, indeed (FIG. 2B, table 2). Although the used compounds included potentially strong axial ligands (e.g. imidazole/acetamide), weak axial ligands (e.g. propanol), and ligands of similar strength as a methionine (e.g. dimethylsulfide), all resulting spectra were similar (FIG. 2C) and reminiscent of wt NiR (FIG. 1A). Ratios of A₄₆₀ over A₆₀₀ were about the same: for example for acetamide-saturated NiR M150G ε₄₅₈=2900, ε₆₀₀=1800 M⁻¹ cm⁻¹, and A₄₆₀/A₆₀₀=1.6 (for wt A₄₆₀/A₆₀₀=1.3) and quite different from M150H (A₄₆₀/A₆₀₀=7.7) and M150T (A₄₆₀/A₆₀₀=0.085). The only exception was the spectrum resulting from the titration with formamide, for which the 460 nm peak shifted to a significantly shorter wavelength and the peak at 360 nm was less intense (FIG. 2C, bottom trace). The remarkably similar spectra observed with all the other ligands suggest that they trigger a similar change in the ligand sphere at the type-1 copper site, without directly binding to the copper.

TABLE 2
Affinity constants of allosteric effectors
for the type-1 site of NiR M150G

| External Ligand | K_{D}^{OX} (mM) |
|---|---|
| dimethylsulfide | 21 ± 4 |
| ethylmethylsulfide | 14 ± 6 |
| formamide | 172 ± 40 |
| acetamide | 71 ± 3 |
| imidazole | 52 ± 3 |
| ethanol | 805 ± 142 |
| propanol | 353 ± 52 |
| acetonitrile | 71.5 ± 7.3 |
| pyridine | 2.6 ± 0.2 |
| 4-methylthiazole | 22 ± 3 |
| nitrite | 157 ± 27 |

All the ligands in this table displayed isosbestic points during titration of the type-1 spectrum. For details see Materials and Methods section.

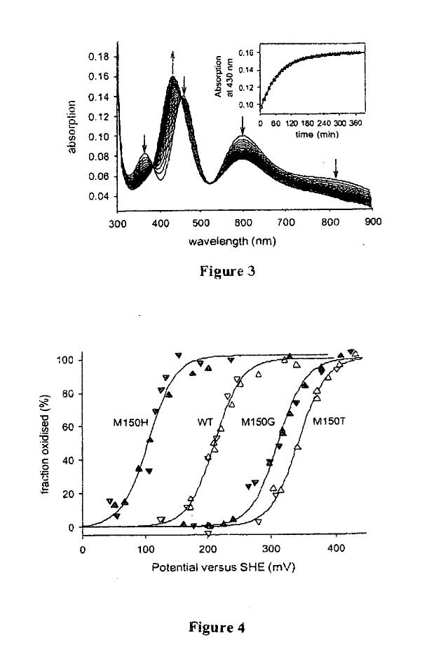

For imidazole bound M1500, a further change of the optical spectrum was observed on a longer time scale. FIG. 3 shows the optical spectrum of NiR M150G-imidazole versus time Spectra of M150G were recorded every 10 minutes in the presence of imidazole (260 mM=5×K_{D}) at 25° C. Arrows indicate the direction of the spectral change. The inset shows the absorption at 430 nm versus time. The solid line in the inset is a fit to a single exponential (yielding a rate of 0.823+0.003 hour⁻¹). After 6 hours the visible spectrum was stable, the 460 nm band had shifted to a significantly shorter wavelength (431 nm) and the A₄₆₀/A₆₀₀ ratio had increased (FIG. 3). When the imidazole was removed by dialysis, the original spectrum of M150G without ligands was observed (results not shown). For other ligands (acetamide, formamide, DMS) no time dependence of the spectrum was observed, not even over a period of weeks at room temperature.

Reduction potential—Reduction potentials were determined to define the driving force for the electron transfer function of the type-1 sites. FIG. 4 shows the results. Downward pointing triangles denote reductive titrations, upward pointing triangles depict oxidative titrations. Filled triangles indicate M150H and M150G, open triangles wildtype NiR and NiR M150T (as indicated in the graph). The solid lines are fits to the Nernst equation for the combined titrations. The midpoint potential of the type-1 site of wt NiR was found to be 213+5 mV versus NHE. For NiR M150H the midpoint potential was extremely low with 104+5 mV. The midpoint potentials of M150T (340+5 mV) and M150G without ligands (312+5 mV) were higher than that of the wt NiR.

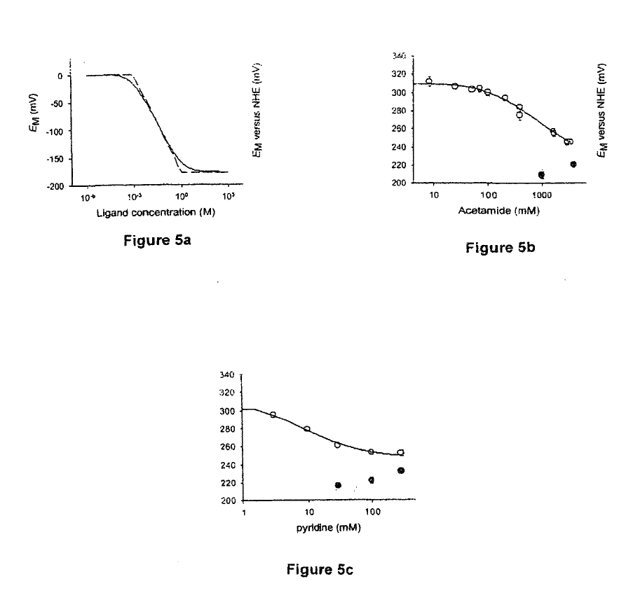

To determine the midpoint potential of NiR M150G with external ligand bound, we measured the dependence of the reduction potential on the ligand concentration for acetamide and pyridine. In FIG. 5 (A), the solid line is a theoretical curve calculated from equation 1 with K_{D}^{α}=1 mM, K_{D}^{RED}=1 M, and E_{M}^{NL}=0 mV, T=298 K. The dashed lines equal the reduction potential of the redox-site without ligand (E_{M}^{NL}=0 mV), saturated with ligand (EML=−177 mV), and the slope in between the dissociation constants. FIG. 5 shows the (B) reduction potential of NiR M150G (open circles) and NiR wt (closed circles) versus acetamide concentration. The thick gray line indicates the reduction potential of wt NiR without ligand. The thin line is a fit of the reduction potential of M150G to equation 1. (C) Reduction potential of NiR M150G and NiR wt versus pyridine concentration, legend as in panel B. FIG. 5A shows the dependence expected for a redox-site for which the binding of a ligand affects the reduction potential. Increasing concentrations of the external ligand lowered the reduction potential (FIG. 5B/C) and the reduction potential levels off at the highest ligand concentrations. However, at these higher concentrations the reduction potential of the wt NiR is significantly increased. Apparently, at the high ligand concentrations, non-specific effects come into play causing an increase in reduction potential of wt NiR, and potentially the smaller decrease in reduction potential of NiR M150G. Therefore, the value obtained for the midpoint potential with ligand bound (E_{M}^{L} see equation 1 and 2) was interpreted as an upper-limit. With acetamide bound to NiR M150G the fitted reduction potential was <225 mV versus NHE, and the K_{D}^{ox} was 157±13 mM. For pyridine bound NiR M150G the reduction potential was <245 mV, and the K_{D}^{ox} was 3.0±0.5 mM. Thus, the midpoint potential of the type-1 site with ligand did not differ substantially from that of the wt NiR.

Activity—The type-1 site of nitrite reductase is essential for catalytic activity; thus, the electron transfer function of type-1 site variants can be assessed by comparison of the catalytic activity of the enzyme variant with that of the wt NiR. Catalytic activity was measured with the physiological electron donor pseudoazurin (table 3 below). NiR M150H had 4 orders of magnitude less activity than the wt NiR. The catalytic activities of NiR M150T and NiR M150G without ligands were one third of that of the wt NiR.

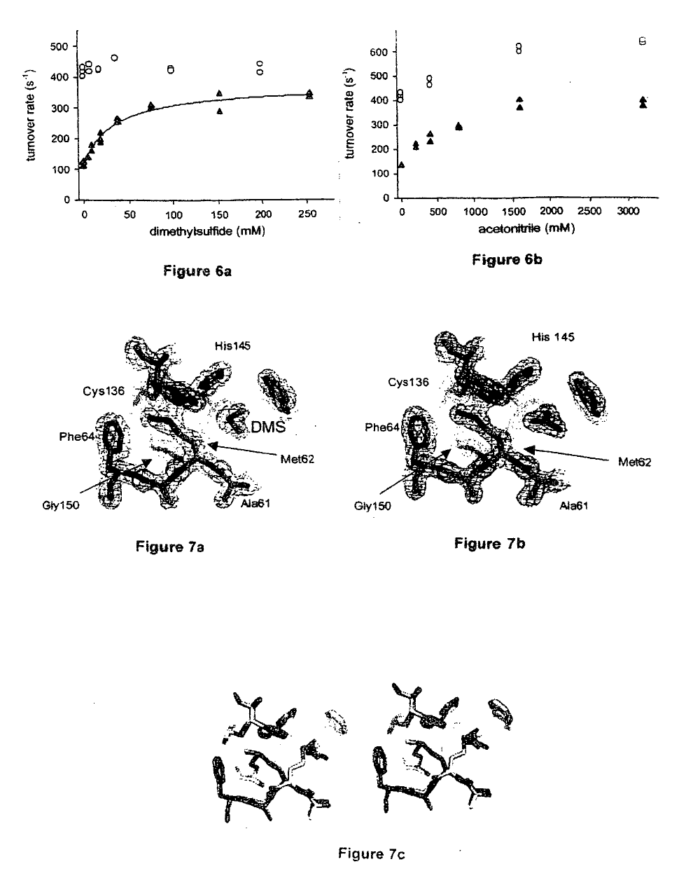

The activity of NiR M150G could be increased by the addition of exogenous ligands (FIG. 6). In FIG. 6, wt NiR: open circles, NiR M150G: triangles, the gray line is a visual reference to the catalytic activity of native NiR in the absence of external ligands. FIG. 6 (A) shows the rate of catalytic turnover versus dimethylsulfide concentration. The thin dark line is the least-squares fit that yielded the apparent dissociation constant and the maximum activity (see Materials and Methods section for details). FIG. 6 (B) shows activity versus acetonitrile concentration. Acetamide and dimethylsulfide (DMS) restored the activity up to the level of wt NiR. The dependence of activity versus ligand concentration could be fit to a one ligand binding equilibrium. The resulting apparent dissociation constant was in all cases higher than the K_{D} for binding to the oxidized type-1 site (table 2 and 3). For ethylmethylsulfide and formamide it was not possible to saturate NiR M150G with ligand; however, activity doubled over a concentration range in which the wt NiR had constant activity (table 3).

TABLE 3
Catalytic activity of NiR variants

| NiR | Saturated with | k_{cat}^{sat} | K_{D}^{app} |
|---|---|---|---|
| variant | external ligand | (s⁻¹) | (mM) |
| WT | — | 416 ± 10 | — |
| M150H | — | 0.099 ± 0.006 | — |
| M150T | — | 126 ± 2 | — |
| M150G | — | 133 ± 6 | — |
| M150G | dimethylsulfide | 373 ± 22 | 34 ± 7 |
| M150G | ethylmethylsulfide | >292 | >73 |
| M150G | formamide | >255 | >4000 |
| M150G | acetamide | 374 ± 28 | 1068 ± 295 |
| M150G | acetonitrile | >390^{A} | ND |

^{A}Also the native NiR increased in activity (see FIG. 6B). A lower limit means that it was not possible to saturate the activity of NiR M150G with ligand.
ND: not determined.

The effects were less straightforward for other compounds because they also influenced the activity of wt NiR. In the case of acetonitrile (FIG. 6B), the wt NiR catalytic activity increased 50% in contrast to an increase of 200% for M150G. Wt and M150G NiR both decreased in activity with imidazole as a ligand. It is noteworthy that structurally different compounds restored the activity to a level not significantly different from that of the wt NiR (table 3).

Structure—Superposition of wt NiR to the DMS and acetamide-bound structures of M150G reveals that these small molecules displace the side-chain of Met62, a residue near the type-1 copper site that is non-coordinating in the wt structure. FIG. 7 shows the crystal structures of the type-1 copper sites Identical views are given for panel A, B and C. Foreground: Ala61-Phe64, His95. Background: Cys136, Trp144, His 145, and Gly150 (mutant) or Met150 (wt). The type-1 copper is a pale sphere. The σ_{A} weighted 2Fo-Fc electron density maps are contoured at 10. FIG. 7A shows the structure of M150G-DMS. FIG. 7(B) shows the structure of M150G-acetamide. FIG. 7A shows a stereo stick representation of wt NiR superimposed with M150G-DMS and M150G-acetamide. The remarkable finding is that in its new position Met62 adopts a conformation that allows its Sδ atom to take up a new position that is similar to the Met150 Sδ position in the native wt structure. DMS and acetamide bind M150G NiR at nearly the same position, roughly 6 Å from the protein surface. The DMS sulfur is 0.46 Å from the Sd atom of Met62 in wild-type NiR. The DMS is in an orientation analogous to that of the Met62 thioether that it displaces and too far from the Cu-atom (4.5 Å) to be a ligand (FIG. 7). Acetamide is slightly further away from the Cu-atom (5.0 Å) and forms hydrogen bonds to two buried water molecules. The acetamide N and O atoms could not be unambiguously assigned. The two buried water molecules are located in a 5 Å deep tunnel that connects to the surface and also is present in the wt and M150G-DMS structures.

The displacement by DMS and acetamide of the Met62 side-chain is accomplished by a 115° rotation of the χ₁ torsional angle, a 25° rotation of χ₂, and a 59° rotation of χ₃. The atomic positions of the Met62 backbone shift only slightly (0.03 Å rms), but the θ torsional angle rotates 27°. As a result of all torsional changes, the Met62 sulfur moves 4.5 Å to bind to the type-1 copper at a position that overlaps that of the Met150 SD in wt NiR (FIG. 7). The geometries of the type-1 sites are almost identical to that of wt NiR (table 4). No other structural perturbations were observed surrounding the type-1 copper site.

TABLE 4
Metal ligand geometry of type-1 sites
in wt Nitrite Reductase and in M150G^{A}

| | native | M150G | M150G |
|---|---|---|---|
| | AfNiR | DMS | acetamide |
| Distances (Å) | | | |
| Axial-Cu | 2.48 | 2.39 | 2.37 |
| 95-Cu | 2.07 | 2.13 | 2.13 |
| 136-Cu | 2.22 | 2.21 | 2.24 |
| 145-Cu | 2.06 | 2.07 | 2.04 |
| Cu-NSN^{B} | 0.64 | 0.57 | 0.63 |
| Angles (°) | | | |
| 136-Cu-95 | 129 | 129 | 126 |
| 136-Cu-Axial | 106 | 97 | 100 |
| Axial-Cu-95 | 89 | 95 | 95 |
| Axial-Cu-145 | 133 | 130 | 129 |
| θ^{C} | 64 | 67 | 71 |

^{A}The numbers 95, 136 and 145 in the left column refer to the N_{δ} of His95, the S_{γ} of Cys136, and the N_{δ} of His145. Axial refers to the S_{δ} of Met150 in the wt NiR (1SJM) and the S_{δ} of Met62 in the M150G structures. Sigma values (standard deviations determined from average values of three monomers in the asymmetric unit) amount to less than 5% for bond angles and less than 3% for bond distances.
^{B}This is the distance between the Cu atom and the NSN plane determined by the ligand atoms of residues His95/Cys136/His145.
^{C}θ is the dihedral angle between the planes through 136-Cu-Axial Ligand and the plane through 136-Cu-145.

A second acetamide molecule is modeled in the active site solvent channel, 7.3 Å from the type-2 copper. In the DMS-bound structure, additional density is present at the substrate binding site of the type-2 copper. This density is modeled as water but may be DMS or a degradation product.

Discussion

Axial Ligand Binding and Spectroscopy—In the crystal structures, the external ligands dimethylsulfide and acetamide do not bind to the Cu atom, but instead they displace Met62 which is coordinated to the type-1 copper. The crystals are grown below pH 5 and data was collected at liquid nitrogen temperature, so a different conformation could prevail in solution at pH 7. This possibility could be excluded by optical spectroscopy.

For type-1 copper sites, the absorbance band at 600 nm originates from n overlap between the copper dx2-y2 and the sulfur orbitals, the 460 nm band originates from pseudo-α overlap between the same orbitals (11). The A₄₆₀/A₆₀₀ ratios in blue copper proteins reveal variations in these overlaps. In the case of a trigonal site, such as in azurins, the dx2-y2 orbital overlaps almost solely with the two histidines and the cysteine, resulting in almost pure n overlap with the cysteine. In tetrahedrally distorted type-1 sites like in the nitrite reductase of Alcaligenes faecalis S-6 (4,31), the d-orbital overlaps with the axial methionine (stronger axial interaction). This change of orientation produces an increased A₄₆₀/A₆₀₀ ratio (32,33), and a shift to shorter wavelengths (11) of both absorption bands. The change in orientation of the dx²-y² orbital can be quantified by the dihedral angle θ between the planes through 136-Cu-Axial and the plane through 136-Cu-145 (see Table 4) (32).

The effects of strong versus weak axial interaction on the optical spectrum of a type-1 copper site can be seen in two examples: NiR M150H (strong) and M150T (weak). The optical spectrum of M150H has a very high ratio of A₄₆₀/A₆₀₀, and peaks that are shifted to shorter wavelengths, while M150T has a very low A₄₆₀/A₆₀₀ ratio. For NiR M150G as purified the A460/A600 ratio is closer to that of the wt NiR than to M150T (31,32,34).

Crystallography of the NiR M150G variant indicates that Met62 and not the added exogenous ligands (DMS/acetamide) bind to the type 1 copper, and optical spectroscopy confirms the crystallographic result. Binding of dimethylsulfide and ethylmethylsulfide to NiR M150G restores the spectroscopic properties to those of wt NiR, which is expected if either these thioether compounds bind directly or alternatively Met62 binds to the Cu atom. For acetonitrile, ordinary alcohols (which mimic threonine), imidazoles (which mimic histidine), acetamide (which mimics glutamine) essentially the same spectra are observed as with dimethylsulfide and ethylmethylsulfide. This result is incompatible with direct binding of these groups to the Cu-atom and rather points to similar Cu-sites in all these experiments. Crystallographic observations correlate well to the solution optical properties. Not only were the ligand-soaked crystals green, also the χ dihedral angles found in the crystal structures, which correlates with the A₄₆₀/A₆₀₀ ratio, are similar for the wt and the two M150G-ligand structures (table 4). All these results indicate that the bound compounds affect the Cu-site structure in the same indirect manner by causing Met62 to bind to the Cu. Thus, the added compounds may be considered as allosteric effectors.

Long-term incubation of NiR M150G with imidazole resulted in spectra indicative of a different axial ligand. A peak shift to shorter wavelengths, accompanied by an increase in the A₄₆₀/A₆₀₀ ratio, suggests stronger axial interaction due to direct copper coordination by imidazole, similar to NiR M150H. Incubation with formamide significantly shifted the A₄₆₀ peak also, possibly indicating that formamide does bind directly, at least partially, to the type-1 copper. Thus, some exogenous ligands may substitute for Met150 by coordinating to the copper.

Midpoint Reduction Potential and Catalytic Activity—The M150T mutation changed the reduction potential by +127 mV with respect to the wt protein, which resembles the shift of +107 mV observed for Rhodobacter sphaeroides NiR M182T (26). For NiR M150G, the change in reduction potential (+99 mV) resembles the change observed for Alcaligenes xylosoxidans NiR M144A (+74 mV (35)) and azurin M121A (+63 mV (27)). For NiR M150H, the shift in reduction potential (−109 mV) is similar to the shift of −100 mV for Alcaligenes denitrificans azurin M121H (36). The observed variations in the reduction potential are in line with the idea that stronger axial interaction lowers the reduction potential of the type-1 site (11). The higher reduction potential of NiR M150T and NiR M150G with respect to the wt may partly explain the lower catalytic activity since it will hinder the electron transfer to the type-2 site. In A. xylosoxidans NiR M144A, the electron transfer rate from the type-1 to type-2 Cu site is indeed tenfold decreased (37). In Achromobacter cycloclastes NiR M150Q (change −127 mV), the electron transfer rate from pseudoazurin to NiR had decreased below the detection limit (23), which is reminiscent of the low activity of our NiR M150H (change −109 mV). Thus, in a qualitative sense the catalytic activities of our NiR variants vary in agreement with the changes in reduction potentials.

To determine the reduction potential of NiR M150G with an allosteric effector bound, we tried to saturate both the oxidized and reduced type-1 sites with ligand (otherwise an average reduction potential with and without ligand bound is measured according to equation 1). Assuming a simple scheme, the K_{D}^{ox} obtained from potentiometric titration should be identical to that obtained from direct ligand titration, which for pyridine is indeed the case. The calculated EM<225 mV versus NHE with acetamide as the allosteric ligand is not significantly different from the value for the wt NiR (213 mV versus NHE). It is unlikely that the reduction potential is much lower than 225 mV, since the catalytic activity (which is a measure of the electron transfer function) with acetamide as the ligand is not distinguishable from that of the wt NiR (table 4). Thus, binding of Met62 to the copper apparently restores the reduction potential of the type-1 site to the wt value and restores electron transfer function as well.

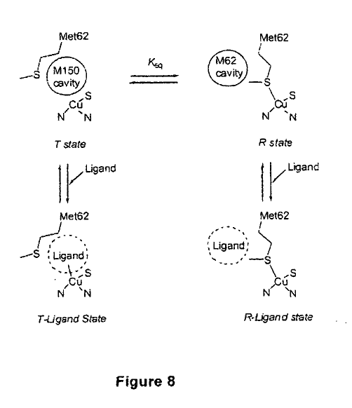

Allosteric Control—The results presented so far can be summarized by the Scheme in FIG. 8. The states at the right and left hand corners at the top of the square depict the type-1 site in the absence of external ligands with Met62 in its native conformation and in a conformation where it binds to the Cu, respectively. In the left conformation the cavity created by the Met150Gly mutation is empty, in the right conformation it is occupied by Met62 and a new cavity is created at the original position of Met62. The two states at the bottom represent similar conformations but with the cavities filled with a “ligand”. The states at the left side of the square are denoted by “T” (from “Tense” denoting an enzymatically less active state), those at the right are denoted by “R” (from “Relaxed” denoting an enzymatically more active state).

The crystallographic results constitute clear evidence for the existence of the R-ligand state. As for the R-state, one may expect its optical spectrum to be identical to that of the R-ligand state since the spectrum appears insensitive to what is present in the Met62 cavity as long as Met62 is coordinated to the Cu. Since the spectrum of the Met150Gly variant in the absence of external ligands is different than when ligand is present, one may conclude that in the former species the Met62 is not coordinated to the Cu. This species is represented by the T-state (top left in FIG. 8). Crystals of the protein in this state could not be obtained so far since components of the crystallization buffer tended to penetrate the protein and to produce an R-ligand state.

The slow conversion of the R-ligand sate in the presence of imidazole into a new state with a strongly differing optical spectrum is an indication for the occurrence of a T-ligand state. Definite proof for the occurrence of this state must await the outcome of further crystallographic experiments, however, as well as further studies of the enzymatic activity of this species. The simplest explanation for the initial formation of an R-ligand state with imidazole is that the “Met62 cavity”, which has a tunnel to the surface, is more accessible than the Met150 cavity, while the subsequent formation of the T-ligand state is much slower but thermodynamically more favourable.

The occurrence of the R-state at this stage is hypothetical; its actual occurrence according to FIG. 8 depends on the values of the various equilibrium constants and the ligand concentration. The important observation in the present context is that conversion of the T-state (top left) with low activity into an R-ligand state with high activity can be effected by adding a ligand to the solution. This is in contrast with earlier work showing that replacement of an (equatorial) ligand by a glycine results in a protein that is inactive even in the presence of external ligands (13,14,20).

The difference between K_{D}^{app} and K_{D}^{ox} (table 2 and 3) we ascribe to binding of the allosteric effector with lower affinity to the reduced type-1 site (FIG. 5). Under the turnover conditions in which the K_{D}^{app} is measured, the type-1 site needs to accept electrons from pseudoazurin and donate them to the type-2 site. If the reduced type-1 site needs to bind the external ligand for efficient electron transfer to the type-2 site, the K_{D}^{app} will be a weighted average between K_{D}^{ox} and K_{D}^{red}.

The only two ligands (imidazole, and formamide) that seem capable of providing a T-ligand state are similar in that both are expected to bind Cu(II) with higher affinity than a thioether group (41). Conversely, alcohols are expected to bind weaker to Cu(II) than a thioether group, and indeed do not bind to the Cu of either azurin M121G or M121A (29). This observation suggests that some of the ligands like ethanol do not bind to the type-1 copper in NiR M150G because the thioether group of the Met62 has greater affinity for Cu(II). When the ligand has higher affinity for the cavity left by Met62, than for Cu(II), then the R-state is also favored over the T-state.

In conclusion, the replacement of the axial methionine in the type-1 site of NiR (Met150) by a glycine creates a protein variant of which the activity can be restored to wt values by allosteric effectors. The presence of a nearby methionine (Met62) that can substitute for Met 150 is crucial for this to occur. As this methionine is conserved in many blue copper proteins (39,40) the conversion of the wt form into a variant that can be activated allosterically appears more generally applicable.

### REFERENCES

1. Malmstrom, B. G., and Leckner, J. (1998) The chemical biology of copper. Curr. Opin. Chem. Biol. 2, 286-292
2. Zumft, W. G. (1997) Cell biology and molecular basis of denitrification. Microbiol. Mol. Biol. Rev. 61, 533-616
3. Wijma, H. J., Canters, G. W., de Vries, S., and Verbeet, M. P. (2004) Bidirectional catalysis by copper-containing nitrite reductase, Biochemistry 43, 10467-10474
4. Kukimoto, M., Nishiyama, M., Murphy, M. E., Turley, S., Adman, E. T., Horinouchi, S., and Beppu, T. (1994) X-ray structure and site-directed mutagenesis of a nitrite reductase from Alcaligenes faecalis S roles of two copper atoms in nitrite reduction. Biochemistry 33, 5246-5252
5. Godden, J. W., Turley, S., Teller, D. C., Adman, E. T., Liu, M. Y., Payne, W. J., and LeGall, J. (1991) The 2.3 angstrom X-ray structure of nitrite reductase from Achromobacter cycloclastes. Science 253, 438-442
6. Libby, E., and Averill, B. A. (1992) Evidence that the type-2 copper centers are the site of nitrite reduction by Achromobacter cycloclastes nitrite reductase. Biochem. Biophys. Res. Commun. 187, 15291535
7. Boulanger, M. J., Kukimoto, M., Nishiyama, M., Horinouchi, S., and Murphy, M. E. (2000) Catalytic roles for two water bridged residues (Asp-98 and His-255) in the active site of copper-containing nitrite reductase. J. Biol. Chem. 275, 23957-23964
8. Boulanger, M. J., and Murphy, M. E. (2001) Alternate substrate binding modes to two mutant (D98N and H255N) forms of nitrite reductase from Alcaligenes faecalis S-6: structural model of a transient catalytic intermediate. Biochemistry 40, 9132-9141
9. Murphy, M. E., Turley, S., and Adman, E. T. (1997) Structure of nitrite bound to copper-containing nitrite reductase from Alcaligenes faecalis. Mechanistic implications J. Biol. Chem. 272, 28455-28460
10. Kataoka, K., Furusawa, H., Takagi, K., Yamaguchi, K., and Suzuki, S. (2000) Functional analysis of conserved aspartate and histidine residues located around the type 2 copper site of copper-containing nitrite reductase. J. Biochem. (Tokyo) 127, 345-350
11. Solomon, E. I., Szilagyi, R. K., DeBeer George, S., and Basumallick, L. (2004) Electronic structures of metal sites in proteins and models: contribution to function in blue copper proteins. Chem. Rev. 104, 419-458
12. Den Blaauwen, T., Van de Kamp, M., and Canters, G. W. (1991) Type-1 and type-2 copper sites obtained by external addition of ligands to a His117gly azurin mutant. J. Am. Chem. Soc. 113, 50505052
13. Jeuken, L. J. C., Ubbink, M., Bitter, J. H., van Vliet, P., Meyer-Klaucke, W., and Canters, G. W. (2000) The structural role of the copper-coordinating and surface-exposed histidine residue in the blue copper protein azurin. J. Mol. Biol. 299, 737-755
14. Jeuken, L. J. C., van Vliet, P., Verbeet, M. P., Camba, R., McEvoy, J. P., Armstrong, F. K., and Canters, G. W. (2000) Role of the surface-exposed and copper-coordinating histidine in blue copper proteins: The electron-transfer and redox-coupled ligand binding properties of His117Gly azurin. J. Am. Chem. Soc. 122, 12186-12194
15. Gorren, A. C., den Blaauwen, T., Canters, G. W., Hopper, D. J., and Duine, J. A. (1996) The role of His117 in the redox reactions of azurin from Pseudomonas aeruginosa. FEBS Lett. 381, 140-142
16. van Pouderoyen, G., Andrew, C. R., Loehr, T. M., Sanders-Loehr, J., Mazumdar, S., Allen, H., Hill, H. A. O., and Canters, G. W. (1996) Spectroscopic and mechanistic studies of type-1 and type-2 copper sites in Pseudomonas aeruginosa azurin as obtained by addition of external ligands to mutant His46Gly. Biochemistry 35, 1397-1407
17. van Pouderoyen, G., den Blaauwen, T., Reedijk, J., and Canters, G. W. (1996) Dimerization of a His117Gly azurin mutant by external addition of 1, omega-di(imidazol-1-yl)alkanes. Biochemistry 35, 13205-13211
18. Vidakovic, M., and Germanas, J. P. (1995) Novel Biological Copper Proteins Through Anion Addition to the Mutant Met121gly of Pseudomonas-aeruginosa Azurin. Angew. Chem., Int. Ed. 34, 1622-1624
19. van Gastel, M., Coremans, J. W. A., Mol, J., Jeuken, L. J. C., Canters, G. W., and Groenen, E. J. J. (1999) The binding of imidazole in an azurin-like blue-copper site. J. Biol. Inorg. Chem. 4, 257-265
20. Wijma, H. J., Boulanger, M. J., Molon, A., Fittipaldi, M., Huber, M., Murphy, M. E., Verbeet, M. P., and Canters, G. W. (2003) Reconstitution of the type-1 active site of the H145G/A variants of nitrite reductase by ligand insertion. Biochemistry 42, 4075-4083
21. Hall, J. F., Kanbi, L. D., Strange, R. W., and Hasnain, S. S. (1999) Role of the axial ligand in type 1 Cu centers studied by point mutations of met148 in rusticyanin. Biochemistry 38, 12675-12680
22. Kataoka, K., Kondo, A., Yamaguchi, K., and Suzuki, S. (2000) Spectroscopic and electrochemical properties of the Met86Gln mutant of Achromobacter cycloclastes pseudoazurin. J. Inorg. Biochem. 82, 79-84
23. Kataoka, K., Yamaguchi, K., Sakai, S., Takagi, K., and Suzuki, S. (2003) Characterization and function of Met150GIn mutant of copper-containing nitrite reductase from Achromobacter cycloclastes IAM1013. Biochem. Biophys. Res. Comm. 303, 519-524
24. Berry, S. M., Ralle, M., Low, D. W., Blackburn, N.J., and Lu, Y. (2003) Probing the role of axial methionine in the blue copper center of azurin with unnatural amino acids. J. Am. Chem. Soc. 125, 87608768
25. Diederix, R. E. M., Canters, G. W., and Dennison, C. (2000) The Met99Gln mutant of amicyanin from Paracoccus versutus. Biochemistry 39, 9551-9560
26. Olesen, K., Veselov, A., Zhao, Y., Wang, Y., Danner, B., Scholes, C. P., and Shapleigh, J. P. (1998) Spectroscopic, kinetic, and electrochemical characterization of heterologously expressed wild-type and mutant forms of copper-containing nitrite reductase from Rhodobacter sphaeroides 2.4.3. Biochemistry 37, 6086-6094
27. Pascher, T., Karlsson, B. G., Nordling, M., Malmstrom, B. G., and Vanngard, T. (1993) Reduction potentials and their pH dependence in site-directed-mutant forms of azurin from Pseudomonas aeruginosa. Eur. J. Biochem. 212, 289-296
28. Romero, A., Hoitink, C. W., Nar, H., Huber, R., Messerschmidt, A., and Canters, G. W. (1993) X-ray analysis and spectroscopic characterization of M121Q azurin. A copper site model for stellacyanin J. Mol. Biol. 229, 1007-1021.
29. Bonander, N., Karlsson, B. G., and Vanngard, T. (1996) Environment of copper in Pseudomonas aeruginosa azurin probed by binding of exogenous ligands to Met121X (X=Gly, Ala, Val, Leu, or Asp) mutants. Biochemistry 35, 2429-2436
30. Tsai, L., Bonander, N., Harata, K., Karlsson, B. G., Vänngard, T., Langer, V., and Sjölin, L. (1996) Mutant Met121Ala of Pseudomonas aeruginosa azurin and its azide derivative: crystal structure and spectral properties. Acta Cryst. D 52, 950-958
31. van Gastel, M., Boulanger, M. J., Canters, G. W., Huber, M., Murphy, M. E. P., Verbeet, M. P., and Groenen, E. J. J. (2001) A single-crystal electron paramagnetic resonance study at 95 GHz of the type-1 copper site of the green nitrite reductase of Alcaligenes faecalis. J. Phys. Chem. B 105, 2236-2243
32. Pierloot, K., De Kerpel, J. O. A., Ryde, U., Olsson, M. H. M., and Roos, B. O. (1998) Relation between the structure and spectroscopic properties of blue copper proteins. J. Am. Chem. Soc. 120, 13156-13166
33. LaCroix, L. B., Shadle, S. E., Wang, Y. N., Averill, B. A., Hedman, B., Hodgson, K. O., and Solomon, E. I. (1996) Electronic structure of the perturbed blue copper site in nitrite reductase: Spectroscopic properties, bonding, and implications for the entatic/rack state. J. Am. Chem. Soc. 118, 7755-7768
34. De Kerpel, J. O. A., and Ryde, U. (1999) Protein strain in blue copper proteins studied by free energy pertubations. Proteins 36, 157-174
35. Ellis, M. J., Prudencio, M., Dodd, F. E., Strange, R. W., Sawers, G., Eady, R. R., and Hasnain, S. S. (2002) Biochemical and crystallographic studies of the Met144Ala, Asp92Asn and His254Phe mutants of the nitrite reductase from Alcaligenes xylosoxidans provide insight into the enzyme mechanism. J. Mol. Biol. 316, 51-64.
36. Kroes, S. J., Hoitink, C. W. G., Andrew, C. R., Ai, J., Sanders-Loehr, J., Messerschmidt, A., Hagen, W. R., and Canters, G. W. (1996) The mutation M121H creates a type-1.5 copper site in Alcaligenes denitrificans azurin. Eur. J. Biochem. 240, 342-351
37. Farver, O., Eady, R. R., Sawers, G., Prudencio, M., and Pecht, I. (2004) Met144Ala mutation of the copper-containing nitrite reductase from Alcaligenes xylosoxidans reverses the intramolecular electron transfer. FEBS Lett. 12, 173-176
38. Fersht, A. (1985) in Enzyme Structure and Mechanism, pp. 256, W.H. Freeman and Company, New York
39. Libeu, C. A. P., Kukimoto, M., Nishiyama, M., Horinouchi, S., and Adman, E. T. (1997) Site-directed mutants of pseudoazurin: explenation of increased redox potentials from X-ray structures and from calculation of redox potential differences. Biochemistry 36, 13160-13179
40. Adman, E. T. (1991) in Advances in Protein Chemistry Vol. 42, pp. 145-197
41. Cowen, J. A. (1993) in Inorganic Biochemistry, pp. 7, VCH Publishers, Inc., New York, Weinheim, Cambridge
42. Nishiyama, M., Suzuki, J., Kukimoto, M., Ohnuki, T., Horinouchi, S., and Beppu, T. (1993) Cloning and characterization of a nitrite reductase gene from Alcaligenes faecalis and its expression in Escherichia coli. J. Gen. Microbiol. 139, 725-733
43. Kakutani, T., Watanabe, H., Arima, K., and Beppu, T. (1981) A blue protein as an inactivating factor for nitrite reductase from Alcaligenes faecalis strain S-6. J. Biochem. (Tokyo) 89, 463-472
44. Kukimoto, M., Nishiyama, M., Ohnuki, T., Turley, S., Adman, E. T., Horinouchi, S., and Beppu, T. (1995) Identification of interaction site of pseudoazurin with its redox partner, copper-containing nitrite reductase from Alcaligenes faecalis S-6. Protein. Eng. 8, 153-158
45. Kukimoto, M., Nishiyama, M., Tanokura, M., Adman, E. T., and Horinouchi, S. (1996) Studies on protein-protein interaction between copper-containing nitrite reductase and pseudoazurin from Alcaligenes faecalis S-6. J. Biol. Chem. 271, 13680-13683
46. Brenner, A. J., and Harris, E. D. (1995) A quantitative test for copper using bicinchoninic acid. Anal. Biochem. 226, 80-84
47. Dutton, P. L. G. (1978) Redox potentiometry: determination of midpoint potentials of oxidation-reduction components of biological electron-transfer systems Methods Enzymol. LIV, 411-435
48. Campbell, I. D., and Dwek, R. A. in Biological Spectroscopy, pp. 220, The Benjamin/Cummings Publishing Company, Inc., London, Amsterdam, Ontario, Sydney
49. Moore, G. R., and Pettigrew, G. W. (1990) Cytochromes c, Springer-Verlag, Berlin, Heidelberg, New York
50. Otwinowski, Z. M., W. (1997) Processing of X-ray diffraction data collected in oscillation mode. Methods Enzymol. 276, 307-326
51. Tocheva, E. I., Rosell, F. I., Mauk, A. G., and Murphy, M. E. P. (2004) Side-On Copper-Nitrosyl Coordination by Nitrite Reductase. Science 304, 867-870
52. Murshudov, G. N., Vagin, A., and Dodson, E. J. (1997) Refinement of macromolecular structures by the maximum-likelihood method Acta Cryst. 53, 240
53. Laskowski, R. A., MacArthur, M. W., Moss, D. S. & Thornton, J. M. (1993) PROCHECK: a program to check the stereochemical quality of protein structures. J. Appl. Cryst. 26, 283-291

1. A method of detecting redox enzyme activity in which an electron transfer enzyme derived from a wild type oxidoreductase having a type-1 copper site is contacted with a substrate for the enzyme to oxidise or reduce the substrate and the enzyme activity is monitored via the activity of an oxidant or reductant, as the case may be, of the type 1 copper site, characterised in that the type 1 copper site has been modified compared to the wild type enzyme by substitution of a copper coordinating residue which coordinates the copper ion of the type 1 site by a residue selected from Gly and Ala, and the enzymatic reaction is carried out in the presence of an allosteric effector, which is a solute molecule which is capable of modifying the activity of the enzyme to allow an electron donating residue of the enzyme to coordinate with the copper ion of the type 1 copper site.

2. The method according to claim 1 in which the enzyme activity is monitored by measuring the current or resistance with electron transfer from the protein to and from electrodes.

3. The method according to claim 2 in which the electron transfer is direct from the protein to an electrode.

4. The method according to claim 2 in which the electron transfer is via a mediator between the protein and the electrode.

5. The method according to claim 1 in which the oxidoreductase is a dissimilatory nitrite reductase.

6. The method according to claim 1 in which the oxidoreductase is an oxidase selected from laccase, ascorbate oxidase, ceruloplasmin and Fet3p.

7. The method according to claim 5 in which the nitrite reductase is NiR from A. faecalis S-6.

8. The method according to claim 7 in which the protein has the 150 Met residue replaced by Gly.

9. The method according to claim 7 in which the substrate is pseudoazurin.

10. The method according to claim 1 in which the solute molecule is selected from metabolites, cholesterol, drugs, hormones, sugars, fatty acids, peptides, alcohols, imidazoles, acetamide and dialkylsulphides.

11. A redox enzyme comprising at least one copper ion and comprising sequence ID1 in which one of the residues His95, Cys136 and Met150 is substituted by a residue selected from Gly and Ala and in which the other of such residues is conserved, in which Met62 is conserved, and in which the remaining residues are identical or up to 50% of them may be conservatively substituted, and/or in which up to 10 residues at the C and/or N terminal of the SEQ ID NO:1 are deleted.

12. The redox enzyme according to claim 11 in which no more than 25%, of the remaining residues are conservatively substituted.

13. The redox enzyme according to claim 11 having SEQ ID NO:2.

14. A nucleic acid encoding the enzyme of claim 11.

15. The nucleic acid according to claim 14 which is dsDNA inserted into a plasmid vector.

16. A microorganism comprising the nucleic acid defined in claim 14.

17. The nucleic acid according to claim 14 having SEQ ID NO:3.

18. A sensor comprising an electrode and, in contact with the electrode, a reaction medium containing:
i) an electron transfer enzyme derived from a wild-type oxidoreductase having a type 1 copper site, that has been modified as compared to the wild-type enzyme by substitution of a copper coordinating residue which coordinates the copper ion of the type 1 copper site by a residue selected from Gly and Ala;
ii) a substrate for the electron transfer protein; and
iii) a solute molecule, or a sample suspected of containing the solute molecule, that is capable as an allosteric effector of modifying the activity of the enzyme to allow an electron donating residue of the electron transfer enzyme to coordinate the copper ion of the type 1 copper site.

19. The sensor according to claim 18 in which the reaction medium further contains a redox mediator.

20. The sensor according to claim 18 in which the electron transfer enzyme is covalently bonded to the electrode.

21. The sensor according to claim 18 which comprises an electrical current comprising current sensing and recording means.

22. The sensor according to claim 18 which comprises several electrodes, each in contact with separate aliquots of the reaction medium, in which the electron transfer enzymes associated with separate electrodes differ from one another in their binding sites for allosteric effectors.

23. The sensor according to claim 22 in which the separate aliquots each contain the same sample suspected of containing a solute molecule, whereby a profile of enzyme activity is determined to identify the solute.

24. (canceled)

25. The sensor according to claim 18 in which the substrate is nitrite.

26. The sensor according to claim 18 in which the solute molecule is selected from metabolites, cholesterol, drugs, hormones, sugars, fatty acids, peptides, alcohols, imidazoles, acetamide and dialkylsulphides.

27. An apparatus comprising a sensor according to claim 18, a counter electrode, an electrical circuit connected to the electrodes and current voltage or resistance measuring device in the circuit.
